(19) United States
(12) Patent Application Publication
(10) Pub. No.: US 2024/0407955 A1
(43) Pub. Date: Dec. 12, 2024

(54) DRESSING FOR TREATING HARD-TO-HEAL WOUNDS AND A PROCESS FOR THE MANUFACTURE THEREOF

(71) Applicant: Warszawski Uniwersytet Medyczny, Warsawa (PL)

(72) Inventors: Malgorzata LEWANDOWSKA-SZUMIEL, Warsawa (PL); Ilona SZABLOWSKA-GADOMSKA, Warsawa (PL); Stefan RUDZINSKI, Warszawa (PL); Marta BOCHYNSKA-CZYZ, Mysiadlo (PL); Tomasz GRZELA, Warszawa (PL); Beata MROZIKIEWICZ-RAKOWSKA, Warsawa (PL)

(73) Assignee: Warszawski Uniwersytet Medyczny, Warsawa (PL)

(21) Appl. No.: 18/697,489

(22) PCT Filed: Sep. 30, 2022
(86) PCT No.: PCT/PL2022/050058

§ 371 Date: Apr. 1, 2024

(30) Foreign Application Priority Data
Sep. 30, 2021 (PL) P.439103
Sep. 30, 2021 (WO) PCT/PL2021/050068

(51) Int. Cl. A61F 13/06; A61L 15/34; A61L 15/58

(52) U.S. Cl. CPC A61F 13/064; A61L 15/34; A61L 15/58

(57) ABSTRACT

A dressing is disclosed for treating hard-to-heal wounds and a process for the manufacture thereof, which may be useful in clinical practice, in particular for treating diabetic foot ulceration.

### FIELD OF THE INVENTION

The invention concerns a dressing for treating hard-to-heal wounds and a process for the manufacture thereof, which may be useful in clinical practice, in particular for treating diabetic foot ulceration.

### BACKGROUND OF THE INVENTION

Mesenchymal stem cells (MSCs), also known as multipotent mesenchymal stromal cells, may be obtained from a number of sources, such as the bone marrow, adipose tissue, and cord blood, being a heterogenous population. In spite of differences, they also have a number of common features, such as an ability to undergo cell division, morphology similar to fibroblasts, ability to adhere to plastic, chondrogenic, osteogenic and adipogenic differentiation potential and presence of surface markers [1, 2]. The main MSCs markers include CD73 (ecto-5′-nucleotidase), CD90 (Thy-1) and CD105 (endoglin) [3]. However, MSCs show no expression of hematopoietic cell markers, such as CD11b (integrin αM), CD14 (TLR4 coreceptor), CD34 (sialomucin), CD45 (protein tyrosine phosphatase receptor type C) and CD79a (membrane glycoprotein MB-1) [2]. The primary criterion used to differentiate MSCs from other cells is the simultaneous presence of CD105, CD73 and CD90 surface antigens with no expression of CD45, CD34, CD14 or CD11b, CD79a or CD19 markers [3]. The stem cells found in the adipose tissue (ADSCs, adipose derived stromal/stem cells) show a number of similarities to the MSCs from other tissues [4]. As with MSCs, ADSCs also have immunomodulatory properties with an ability to differentiate into mesodermal cells: chondro-, osteo-, adipoblasts [2, 5], and also other specialized cells, such as skeletal muscle myoblasts [6] or even neurons [7, 8].

In terms of the future applications of MSCs, it is important that they are able to suppress inflammation. The effect may derive from blocked influx of inflammatory cells which would disrupt repair if they were retained and active in the tissue for too long. An advantage of MSCs over topical administration of cytokines, for example, is the feedback between MSCs and other cells so that MSC signaling can adapt to the changing situation, such as suppression of inflammatory response in the first stage of regeneration and production of stimulators of cell proliferation and differentiation in the subsequent stage. Therefore, MSCs may have a positive effect on tissue regeneration even if they are not involved in the formation of the new repaired tissue by themselves. In addition, when stimulated by selected factors, MSCs may increase their ability to improve tissue regeneration. A massive advantage of MSCs is that they may be obtained from various sources at any stage of the patient's life. Recently, the adipose tissue and ADSCs, that is, adipose-derived mesenchymal stem/stromal cells obtained from the tissue, have become a popular source of MSCs. The advantage of ADSCs is availability and abundance in the adipose tissue, and they are relatively easy to obtain. ADSCs typically do not require intensive proliferation in the laboratory to prepare the required quantity considered a therapeutic threshold. Owing to the wide availability of ADSCs, allogeneic therapies based on ADSCs are possible. Therefore, the therapeutic effect of ADSCs may be achieved owing to their differentiation ability, the immunomodulatory effect and ability to regulate processes in their vicinity by secreting appropriate cytokines and direct contact with other cells.

Methods for the isolation of mesenchymal stem cells from the adipose tissue have been reported in the art that yield a heterogenous Stromal Vascular Fraction (SVF) (e.g. [9, 10]) from which ADSCs are derived.

Mesenchymal stem cells are currently the most frequently used and uncontroversial source of cells used in rapidly developing regeneration medicine, with the adipose tissue being a particularly favorable source of these cells.

Chronic ulcers (CUs) (also referred to herein as an ulceration) are defined as disruption of tissues, mainly of the skin, that does not heal spontaneously. In detail, an ulceration (chronic wound, hard to heal wound) is any wound that persists chronically (regardless of its etiology), in which, as a result of the breakdown of pathologically altered (necrotic) tissue fragments, there is a cavity and the exposure of more profound tissues in the form of a crater. This applies when a wound is characterized by exudate, necrotic lesions, biofilm, undergoes an increase in the size of the wound from the 3rd day after its appearance. The aforementioned characteristic concerns all types of wounds, especially with underlying systemic diseases significantly affecting the prolongation of the wound healing process. They include diabetes, chronic venous insufficiency, atherosclerotic ischemia, wounds accompanying the process of neoplastic proliferation, or autoimmune diseases, as well as conditions resulting from local tissue injury, including pressure ulcers, trauma, local infection and burns. An important common denominator of CUs is the presence of local tissue ischemia related to the damage of all types of vessels. CUs affect more than 1% of the general population and up to 4% of people over the age of 80 years. About 10% of patients affected by diabetes present CUs in the form of Diabetic Foot Ulcers (DFUs). These are chronic pedal wounds associated with infection, pain, skin discoloration, and occasional bleeding. This is due to accumulating blood vessel injuries, which render the tissues more prone to damage by minor pressure. DFUs, and CUs in general, significantly decrease patients' quality of life, often culminating in depressive disorders. CUs increase the risk of chronic infection and amputation.

CUs result from combined angiopathy and neuropathy, causing reduced sensation in the affected tissue (Tahergorabi and Khazaei, 2012, Int J Prev Med, 3 (12), 827-38).

When unnoticed, trauma in the affected area without sufficient blood flow to ensure wound healing provides an optimal milieu for injury and infection, resulting in chronic skin ulceration. In addition, CUs may originate from skin burns, trauma, prolonged physical pressure or surgery. The current treatments of CUs have limited efficacy, and they include wound dressings, topical use of growth factors, application of animal dermo-epidermic substitutes, exudate removal, hyperbaric oxygen therapy (Kessler et al., 2003, Diabetes Care, 26 (8), 2378-82) and debridement of necrotic tissues. Because of their xenogenic origin, the used porcine dermo-epidermic substitutes (Apligraf®, Organogenesis Inc., USA) induce transient inflammatory-like reactions that facilitate healing, but they are rapidly rejected. In light of these facts, a large number of studies focus on the novel biological therapies that may be used to treat CUs.

Adipose-Derived Stem Cells (ADSC) are a component of the human adipose tissue that produces large amounts of most of the factors needed for efficient wound healing.

ADSCs are easily isolated and expanded in culture through lipo-aspiration or fat sampling. ADSCs are classified based on the specific criteria from the International Federation of Adipose Therapeutics and Science (IFATS) (Bourin et al., 2013, Cytotherapy, 15 (6), 641-8) and they are currently one of the most studied cells for regenerative cell therapy applications (Si et al., 2019, Biomed. Pharmacother., 114:108765).

ADSCs are considered an encouraging treatment option for chronic wounds since numerous preclinical studies in animals demonstrated that ADSCs transplanted in the ulcer environment facilitate wound repair (Gdelkarim et al., 2018, Biomed. Pharmacother., 107, 625-633) through collagen/matrix secretion and deposition, growth factor secretion, angiogenesis and re-epithelization. Even though multiple intramuscular injections of ADSCs in liquid suspension might be a favorable therapeutic option in patients with DFU (Lee et al., 2012, Circ. J., 76 (7), 1750-60; Bura et al., 2014, Cytotherapy, 16 (2), 245-57), their physical/temporal stability within the wound bed remains a critical issue to address. Indeed, it has been reported that injected ADSCs are locally unstable, display high motility and ultimately spread to sites far from the wound (Zhao et al., 2016, Cytotherapy, 18 (7) 816-27).

Patent application WO 2016/209166 discloses a method for skin regeneration using stem cells deposited on a porous material as a result of their culturing in the presence of such a matrix. Patent application EP3795184A1 discloses dressings in the form of bioresorbable foam made of collagen and/or gelatin impregnated with autologous ADSCs, intended for treating CUs, and in particular for skin regeneration and healing in patients with DFU.

Patent application US 2018/0117217 discloses a sheet for alleviating epidermolysis bullosa comprising mesenchymal stem cells suspended in a hydrogel. However, said product is not suitable for treating CU as it has been shown in subject application. In particular, it does not allow for sufficient ADSCs migration into wound tissue. Moreover, it requires using of stimulated ADSCs.

### SUMMARY OF THE INVENTION

The object of the invention is to provide a dressing useful for treating CU, in particular DFU, that would be suitable for long-term storage at low temperatures, would use the properties of ADSCs that facilitate wound healing and would not be limited to autologous applications. Such a dressing would enable easier and more widespread use in clinical practice. A specific object of the invention is to provide a type of the dressing that would enable cell proliferation on the dressing, both during its preparation and also in conditions typical of the wound environment.

Another objective of the invention is to provide a type of dressings that delivers the unstimulated ADSCs (not exposed to any physical, hypoxic or inflammatory factors during the manufacture process) to the wound environment and allows ADSCs to migrate into the wound site. Another object of the invention is to provide a type of the dressing so that ADSC viability after the freezing process and subsequent storage at −80° C. (for at least 24 h) is at least 50%.

Another object of the invention is to provide a type of the dressing that simultaneously ensures ADSC migration from the dressing in the model wound environment. Advantageously, at least 80% of the live ADSCs found in the dressing migrates to the wound environment compared to the pool of live cells after thawing.

Another object of the invention is to provide a type of the dressing that simultaneously ensures wound healing in the in vitro model represented by scratch closure assay. Advantageously, the scratch area decreases by at least 80% 72 h after a test using the dressing.

Unexpectedly, this comprehensive technical effect has been achieved in the said invention.

The subject of the invention is a dressing and a process for the manufacture thereof as specifically defined in the appended claims.

In connection with the present invention, “polymer containing polyester” is the term given to the polymer that contains the ester functional group (COO—) in every repeat unit of their main chain. Polyethylene terephthalate (or poly(ethylene terephthalate), PET, PETE, or the obsolete PETP or PET-P), is the most common thermoplastic polymer resin of the polyester family. In particular, “polymer containing polyethylene terephthalate (PET)” is the term given to the polymer that consists of polymerized units of the monomer ethylene terephthalate, with repeating (C₁₀H₈O₄) units.

In connection with the present invention, “polymer containing polyurethane” is the term given to the polymer that contains NHCOO (urethane) groups in every repeat unit.

In connection with the present invention, “silicone gel” is the term given to the substance in which silicone, also called polysiloxane, is any of a diverse class of fluids, resins, or elastomers based on polymerized siloxanes, substances whose molecules consist of chains made of alternating silicon and oxygen atoms.

In connection with the present invention, “ADSCs” is the term given to adipose derived stem/stromal cells.

In connection with the present invention, “unstimulated ADSCs” is the term given to ADSCs cells which have been not exposed to any physical, hypoxic or inflammatory factors.

In connection with the present invention, “ADSCs seeded on the dressing material” is the term given to ADSCs cells which have been seeded directly on the surface of dressing material in the manner routinely used in culture of adherent cells in culture media which does not contain any animal-derived components (Xeno-free media), where the standardized supplement (attachment fluid) designed for the attachment of cells under serum-free and xeno-free culture conditions is used to replace bovine/calf serum in facilitating the attachment and spreading of cells on the dressing surface with no need of using any additional hydrogel support. Such fluid supplement is a standard additive to xeno-free culture media and may contain extracellular matrix proteins: fibronectin, collagen, laminin, elastin, vitronectin.

In connection with the present invention, “XenoFree medium” is the term given to the medium that is suitable for the in vitro expansion and culturing of the MSC cells, including ADSC and does not contain nor requires the addition of any animal-derived components such as, but not limited to, FBS/FCS (Fetal Bovine Serum/Fetal Calf Serum). The process of manufacturing Xeno-free medium is highly controlled and provides more batch-to-batch consistency which gives better control over the repeatability of the final product.

To facilitate understanding the essence of the invention, the specification is illustrated by the appended figures and the examples below.

### BRIEF DESCRIPTION OF THE FIGURES

FIG. 1 shows cell proliferation determined using the Presto Blue assay that measures cell metabolic activity for ADSC cultures seeded on dressings. The ratio of the sample fluorescence to the blank from the respective timepoints was calculated to determine the cells amount foldchange. The results are expressed as means of several (at least three) technical replicates±SD (standard deviation).

FIG. 2 shows ADCS culture on dressings:

a-e—Nikon TE2000-U light microscope images: a—UrgoTul, b—Vliwaktiv, c—MepitelOne, d—Mepilex, e—Atrauman silicone. Narrow (blue) arrows indicate ADSCs found on the dressing. Wide (yellow) arrows indicate the dressing.

f-i—HITACHI TM3000 scanning electron microscope images that confirm the presence of cells on the dressings: f—UrgoTul dressing with ADSCs, g—Mepilex dressing with ADSCs, h—Vliwaktiv dressing with the ADSCs, i—Atrauman silicone dressing with ADSCs.

FIG. 3 shows images of dressings before freezing and after thawing. The arrows indicate sites of cell growth. a—MepitelOne before freezing, b—MepitelOne after thawing, c—UrgoTul before freezing, d—UrgoTul after thawing, e—Atrauman silicone before freezing, f—Atrauman silicone after thawing. Storage conditions for the frozen dressings: −196° C., for at least 24 h.

FIG. 4 shows images based on microscope observations after the dressings were applied on a model wound environment. Narrow (blue) arrows indicate sites in which cells are still found on the dressing and wide (yellow) arrows indicate sites in which cells spread from the dressing to the model wound environment which consists of fibrin glue mixed with a wound fluid. a-b—UrgoTul 3 days after the dressings were applied, c-d—MepitelOne 3 days after the dressings were applied, e-f—MepitelOne 7 days after the dressings were applied, g—UrgoTul 7 days after the dressings were applied, h—images of the MepitelOne dressing immediately after thawing and application on the model wound environment, day 1, i—images of the MepitelOne dressing 3 days after thawing and application on the model environment, j—images of the MepitelOne dressing 7 days after thawing and application on the model environment, k—images of the glue after removing the MepitelOne dressing from the model wound environment; position in which the edge of the dressing was located; sites of cell migration from the dressing can be seen, however, the whole cell coat previously located on the dressing which remained on the glue after being removed from the model is also seen; and another image of the central location of the dressing on the glue (not the edge), l—Atrauman silicone 3 days after the dressings were applied, m—Atrauman silicone 7 days after the dressings were applied, n—images of the Atrauman silicone dressing 3 days after thawing and application on the model environment, o—images of the Atrauman silicone dressing 7 days after thawing and application on the model environment, p—images of the glue after removing the Atrauman silicone dressing from the model wound environment, sites of cell migration from the dressing can be seen, r—images of the glue after removing the Atrauman silicone dressing from the model wound environment, to visualize the site of cell migration from the dressing, the cells were treated with MTT, formation of formazan crystals from MTT confirms cell viability (these procedures are based on the reduction of tetrazolium by mitochondrial dehydrogenase enzymes, which is carried inside living cells), s—images of the glue after removing the thawed Atrauman silicone dressing from the model wound environment, to visualize of site of cell migration from the dressing the cells were treated with MTT, formation of formazan crystals from MTT confirms cell viability (these procedures are based on the reduction of tetrazolium by mitochondrial dehydrogenase enzymes, which is carried inside living cells).

FIG. 5 shows: a—cell proliferation using the Presto Blue assay that measures metabolic activity of fibroblast cells (nHF) before and after the test. The lower fluorescence intensity level for nHF+UrgoTul+ADSC is due to the fact that lower stiffness of UrgoTul dressing caused difficulties in management in in vitro cultures. During the operators' manipulations to remove the dressings before fluorescence assessment the surface of well in which fibroblasts were growing was scratched and some of cells were torn off and removed with the dressing; b—a chart showing the scratch closure rate: control tests, nHF without the dressing; lowest rate, 20.33% of the original scratch area remained. The scratch closed most rapidly in the variant with ADSCs seeded on MepitelOne, wherein the surface was 100% closed after 72 h of observation; the scratch surface where ADSCs were seeded on the UrgoTul dressing closed in 89.7% of the original area; the scratch surface where ADSCs were seeded on Atrauman silicone closed in 97%.

FIG. 6 ADSC proliferation after long term storage in liquid nitrogen

a—A chart shows the Presto Blue assay results for ADSC seeded on Mepitel One dressing and was stored frozen in liquid nitrogen for 14 and 54 days. After thawing the Presto Blue assay was performed on the dressings and repeated daily for 7 days.

b—Mepitel One, stored in liquid nitrogen for 1 year, 24 h after thawing, c—Mepitel One, stored in liquid nitrogen for 1 year, 7 days after thawing.

FIG. 7 Comparative migration into the wound environment

Images based on microscope observation after the dressings were applied on the model wound environment. The cells form the dressing from example 1 (i.e. exemplary embodiment of subject invention) showed much higher migration than from the dressing prepared according to the patent application US 2018/0117217; a—images of the glue after removing the Atrauman silicone dressing from the model wound environment, to visualize the site of cell migration from the dressing the cells were treated with MTT, formation of formazan crystals from MTT confirms cell viability (these procedures are based on the reduction of tetrazolium by mitochondrial dehydrogenase enzymes, which is carried inside living cells), b—images of the glue after removing the dressing prepared according to the patent application US 2018/0117217 from the model wound environment, to visualize the site of cell migration from the dressing the cells were treated with MTT, formation of formazan crystals from MTT confirms cell viability (these procedures are based on the reduction of tetrazolium by mitochondrial dehydrogenase enzymes, which is carried inside living cells).

### EXAMPLE 1. MANUFACTURE OF DRESSINGS COATED WITH ADSCS

All operations related to preparation of dressings and cells as well as cultures are performed in sterile conditions in a laminar flow cabinet.

1. Manufacture of the Dressing Material for Culture with Cells.

Using a sterile scalpel or scissors, the dressing material is cut into fragments that fit the culture vessel (e.g. 1.2 cm×1.2 cm fragments are cut for a 24-well plate) and placed in a closed sterile container. Fibronectin solution is prepared in a separate vessel by mixing fibronectin with DPBS w/o Ca, Mg at a 1:100 ratio. The prepared solution is poured on the dressing material so that it is completely submerged in the fibronectin solution and placed at 37° C. for incubation for 30-60 minutes. After the end of incubation, the fibronectin solution in which the dressing material was incubated is aspirated and washed with fresh DPBS. Such a dressing material is transferred into a non-adherent plate (24-well plate) (one fragment per one well on the plate). A silicone separator, onto which the cell suspension will be applied dropwise, is placed on each dressing. Owing to the separator, the cell suspension is retained on the dressing material until the cells adhere to its surface.

### 2. Preparation of ADSCs for Culture on the Dressing Material.

After storage in liquid nitrogen, ADSCs were thawed at 37° C. and transferred to Falcon tubes with an appropriate growth medium. The suspension was centrifuged at 5 min, 350×g, 22° C. After centrifugation, the supernatant was removed and a fresh volume of the growth medium previously heated to 37° C. was added to the remaining pellet; subsequently, their density was determined using an ADAM MC cell counter. The optimum density of ADSCs seeded in a T75 bottle is 0.5-1.5×10⁶ cells.

The incubation conditions were maintained at 37° C. and 5% CO₂. The cells were cultured in a growth medium specific for the cells (XenoFree medium). The cultures were placed in an incubator and grown until confluence of approx. 85%, and then passaged or used for preparing an experiment. To this end, the medium should be removed, and cells washed with DPBS w/o Ca, Mg previously heated to 37° C. After removing DPBS, Accutase heated to room temperature was poured on the cells. The culture vessel with Accutase was placed at 37° C. for 5 minutes (incubation time with Accutase can be increased to 20 minutes, and the degree of cell detachment was tested every 3-5 minutes). To harvest the cells, an adequate volume of the growth medium is added to the culture vessel, pipetted several times and the whole culture suspension is collected into a sterile test tube. The cell suspension is centrifuged for 5 minutes at 350×g, 22° C. The supernatant is removed after centrifugation. A fresh volume of the medium heated to 37° C. is added to the cell pellet and pipetted several times to obtain homogeneous cell suspension to be counted. Cell density as counted should be between 1.25× 10⁶ and 4.0×10⁶ cells/mL. Cells between passages 2 and 4 were collected for subsequent stages.

### 3. ADSC Culture on the Dressing Material.

The expanded cells prepared according to section 2 were applied on a previously prepared (see section 1) dressing material. Optimum cell density for a dressing material fragment of 1.2 cm×1.2 cm is 2.5×10⁵ per 200 μL. Such cell density provides most efficient settlement, highest survival rate during freezing/thawing procedures and after exposure to the harmful wound environment and might increase the effectiveness of the therapeutic potential of the dressing. A serum-free culture medium such as XenoFree should be used (cf. for example US20130136721, WO2015008275A1) for culturing human mesenchymal stem cells, such as for example Nutristem (Biological Industries Genos). The culture vessel is placed at 37° C., 5% CO₂ for at least 3 h so that the cells can settle on the dressing material. After that time, the separators are removed from the dressings and the wells with the dressings are filled up with the medium to adequate volume according to the recommended specification for the culture vessel. To avoid a situation where the dressings float on the surface of the medium, the dressings may be immobilized with weights.

Among the commercially available dressing materials, several different dressing materials (see Table 1) were selected after preliminary tests and evaluations, and were coated with ADSCs according to the previous description and subsequently further tested to obtain the dressing of the invention.

TABLE 1
Preliminarily selected dressing materials

| Trade name/ | | |
|---|---|---|
| Manufacturer | Structure and components of the material | Literature reference |
| UrgoTul/ | Polyester mesh impregnated with hydrocolloid | EP1143895B1 |
| Urgo. | particles dispersed in a petrolatum matrix. The | WO 00/16725 |
| | mass contains a hydrocolloid | |
| | (carboxymethylcellulose), paraffin oil, | |
| | petrolatum and carrier polymers | |
| MepitelOne/ | Polyurethane membrane with regular oval | EP2934611A1 |
| Mölnlycke | holes coated with silicone gel on one side | WO2014/096273 |
| Health Care | | |
| AB | | |
| Mepitel/ | Translucent membrane obtained from | https://www.molnlycke.pl/produkty- |
| Mölnlycke | polyamide fibers coated with silicone gel on | i-rozwiazania/mepitel/ |
| Health Care | both sides | |
| AB (*) | | |
| Mepilex/ | Polyurethane foam coated on the outside with | https://www.molnlycke.pl/produkty- |
| Mölnlycke | a semipermeable polyurethane membrane | i-rozwiazania/mepilex/ |
| Health Care | whose surface is coated with silicone gel on | |
| AB (*) | one side | |
| Vliwaktiv/ | Absorbable dressing with activated carbon, | https://www.lohmann- |
| Lohmann & | with a nonwoven substrate having a 3-2-1-2-3 | rauscher.com/pl- |
| Rauscher (*) | 1 structure, where: 1 - activated carbon with | pl/produkty/opatrywanie- |
| | rayon, 2 - cellulose, 3 - polypropylene | ran/specjalne-zaopatrywanie- |
| | membrane, with the substrate coated on the | ran/vliwaktiv/ |
| | outside with rayon and polyamide | |
| Atrauman | Polyethylene terephthalate (PET) mesh | https://www.hartmann.info/en- |
| Silicone/ | coated on both sides with silicone gel, based | us/our-products/wound- |
| PAUL | on polydimethylsilicosan | management/wound-contact- |
| HARTMANN | | layers/silicone-contact- |
| AG | | layers/atrauman%C2%AE- |
| | | silicone#products |

(*) comparative example

### EXAMPLE 2. EVALUATION OF ADSC VIABILITY AND PROLIFERATION ON THE DRESSINGS

Functional tests to evaluate ADSC viability and proliferation on the dressings were performed for the dressings obtained according to Example 1 using various dressing materials by in vivo staining. The dressings on which cells were present and growth occurred were used for the test. The Presto Blue assay was performed in three technical replicates. Presto Blue is an assay to measure cell metabolic activity. The test uses resazurin conversion to resorufin that occurs in live cells. Resazurin is blue and it is able to enter cells. Live cells convert resazurin to resorufin which is red and shows fluorescence.

To perform the test, working solution of Presto Blue is prepared in the medium for all wells at a 1:10 ratio. The cells are washed once with DPBS w/o Ca, Mg heated to 37° C. An appropriate volume of the working solution of Presto Blue is added to a 0.5 mL well of a 24-well plate and incubated for 2 h at 37° C.

After incubation, the medium from each well, such as 100 μL each, is transferred to a 96-well plate dedicated for fluorescence reading. Fluorescence was read at excitation parameters of 540 nm (±10 nm) and emission at 620 nm (±10 nm). A FLUOstar OPTIMA reader was used to measure fluorescence.

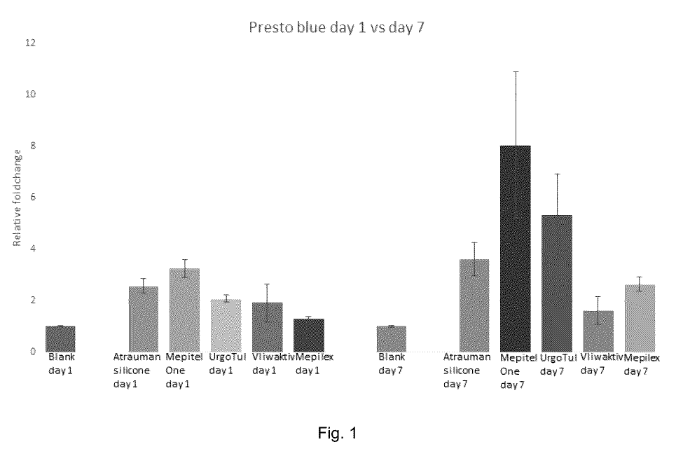

Results of the Presto Blue assays for the dressings prepared using the Mepilex, Vliwaktiv, UrgoTul, MepitelOne and Atrauman silicone materials are shown in FIG. 1. The results are expressed as means of several (at least three) technical replicates±SD (standard deviation).

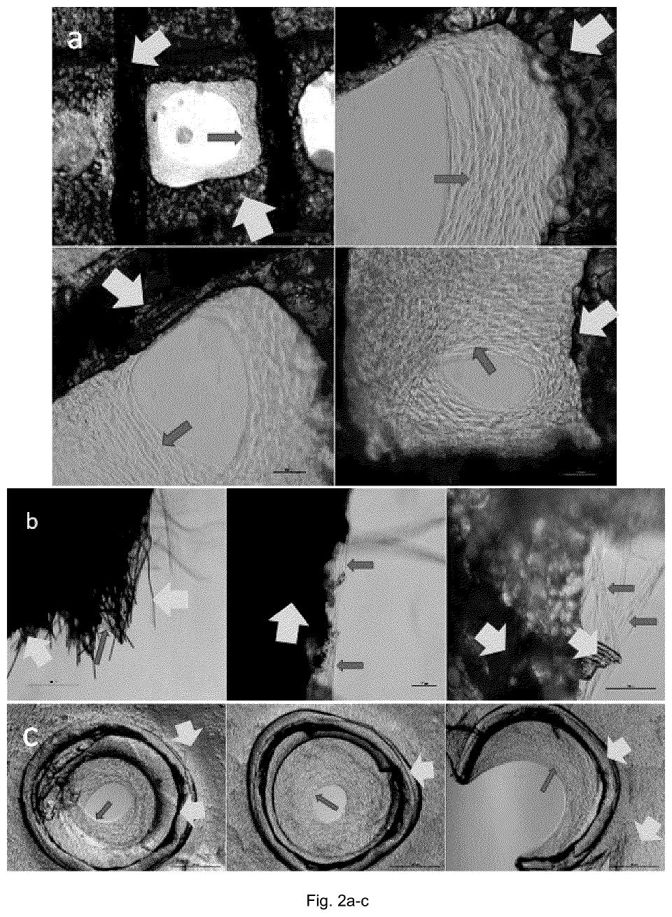

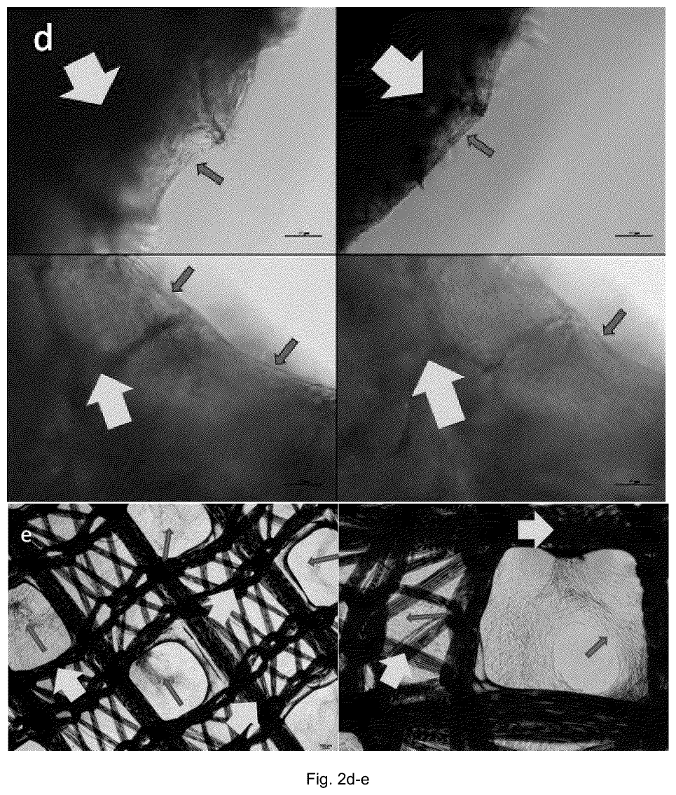

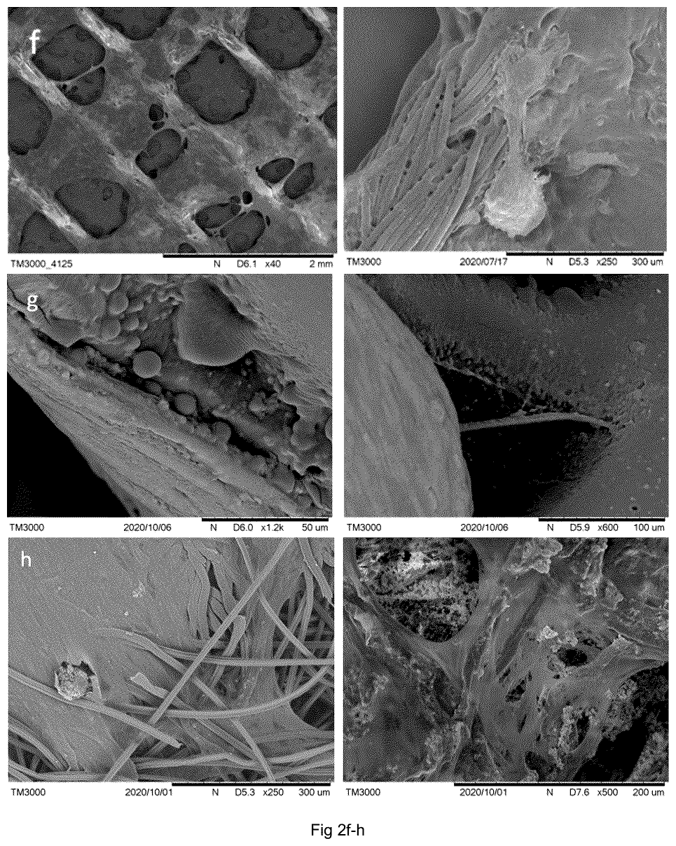

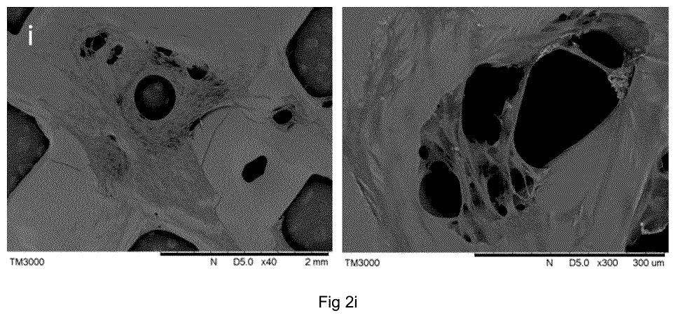

In addition, the dressings prepared using the materials listed in Table 1 were observed under the microscope (light microscope and electron microscope). The results are shown in FIG. 2. The evaluation of ADSC viability and proliferation on the resulting dressings confirmed ADSC adhesion and proliferation on all the materials except for the Mepitel and Vliwaktiv dressing materials, and in effect the dressing prepared using said material was excluded from further testing.

### EXAMPLE 3. DRESSING FREEZING TEST

Freezing the Dressings with the Cells.

Wound dressings prepared in accordance to Example 1 were subjected to standard protocol for cell freezing that can be carried out in accordance to GMP (Good Manufacturing Practice) regulations. Selected dressings were rinsed with DPBS w/o Ca, Mg, and subsequently gently placed in a 2 mL cryotube using tweezers and a serum-free cryoprotectant solution-w/o FBS/FCS addition was poured. The cryotubes were placed in a deep freezer at −80° C. for at least 24 h and subsequently transferred into a liquid nitrogen container.

### Thawing the Dressings

The cryotubes were removed from the dewar/−80° C. freezer and placed in a heating block at 37° C. for 5 minutes. Subsequently, a fresh volume of the medium heated to 37° C. was added to the cryotube. The dressing was transferred to a sterile culture vessel filled with a fresh volume of the medium. The vessel was placed on a rocker with slight shaking for 5 minutes to remove any residual cryoprotectant. Subsequently, after removing the solution, a fresh volume of the culture medium was added to set up cultures or the dressings were used for further testing.

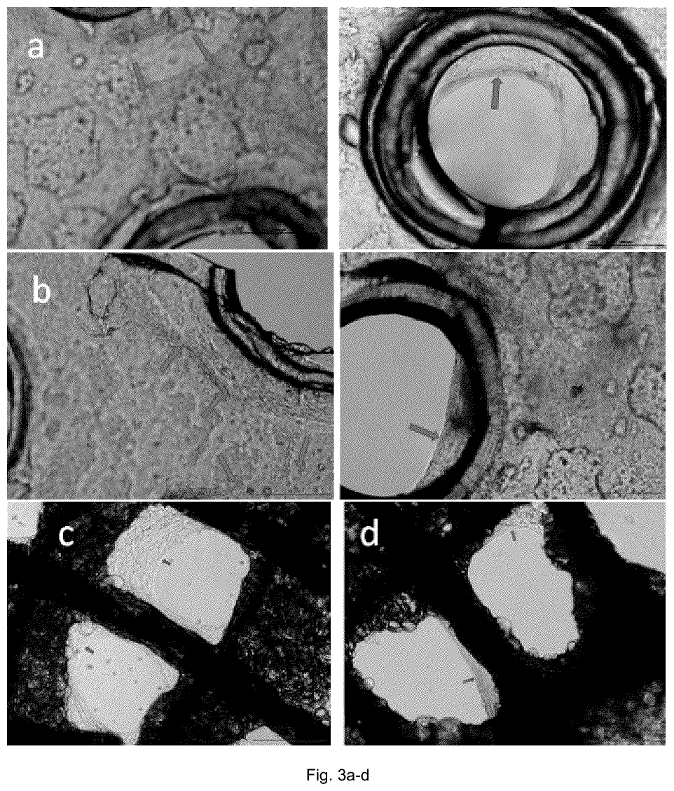

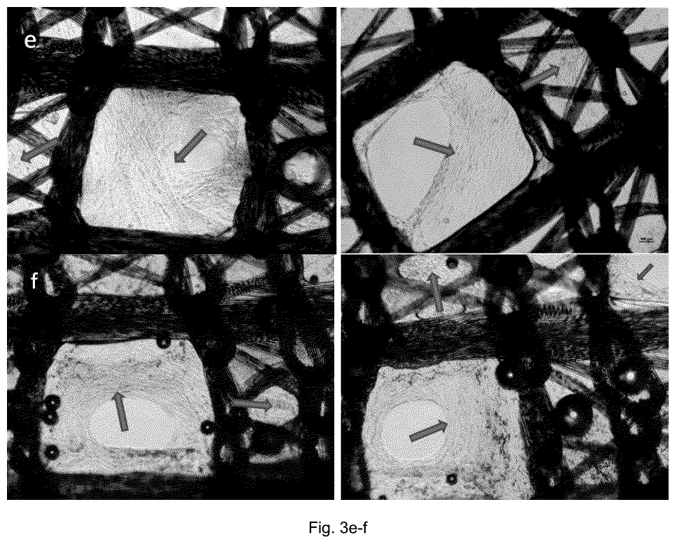

The dressings prepared using the UrgoTul, MepitelOne, Mepilex and Atrauman silicone materials were used for testing. A series of microscope observations was conducted in the new cultures after thawing to evaluate the cell status. The results are shown in FIG. 3. The blue arrows indicate sites of cell growth. a—MepitelOne before freezing, b—MepitelOne after thawing, c—UrgoTul before freezing, d—UrgoTul after thawing, e—Atrauman silicone before freezing, f—Atrauman silicone after thawing. Storage conditions for the frozen dressings: −196° C., at least 24 hours.

Unexpectedly the GMP protocol for freezing cells successfully retained viable cells on dressings proving no need for excessive means i.e. CryoMACS, CryoVac etc. Sites with live cells were seen in the microscope observation of the dressings (UrgoTul, Atrauman silicone and MepitelOne) after thawing. Freezing and thawing are the processes that may result in lower cell viability and performance: here it is also seen as reduced areas with live cells before and after freezing and areas where zones of dead cells are seen.

No cell recovery was seen after thawing for the Mepilex dressing. This could have been caused by the spatial structure of the dressing (foam) which strongly absorbs solutions. The solution could not be completely removed when the dressing was washed in DPBS before the freezing process so the cryoprotectant was diluted and this resulted in cell membranes being broken apart by crystals from PBS in the dressing and cell death occurred.

Due to the negative result of the freezing test of the dressing obtained from the Mepilex material, said dressing was eliminated from further testing.

### EXAMPLE 4. ADSC MIGRATION TEST FROM DRESSINGS IN A MODEL WOUND ENVIRONMENT

### Model Wound Environment

To obtain a model that imitates the wound environment, fibrin glue mixed with a wound fluid obtained from 3 patients with diabetic wounds was used so that the total protein concentration in the final solution was equal in all tests. The wound fluid was DPBS w/o Ca, Mg to which a specimen (scrapings) obtained during cleansing of a diabetic wound was collected.

An experiment was performed in which the response was observed of cells attached to the dressings and placed on a layer of fibrin glue mixed with the wound fluid to imitate the wound environment of individuals with hard-to-heal diabetic wounds. Unexpectedly, very strong cell migration from three dressings on the model wound environment was seen. The observations were made in the dressings obtained from the UrgoTul, MepitelOne and Atrauman silicone materials, derived directly from cultures and from thawed dressings (freezing and thawing was performed according to Example 3).

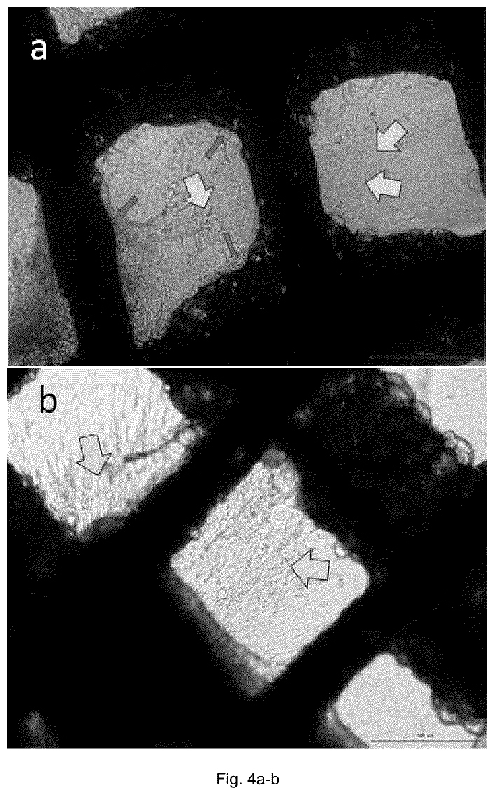

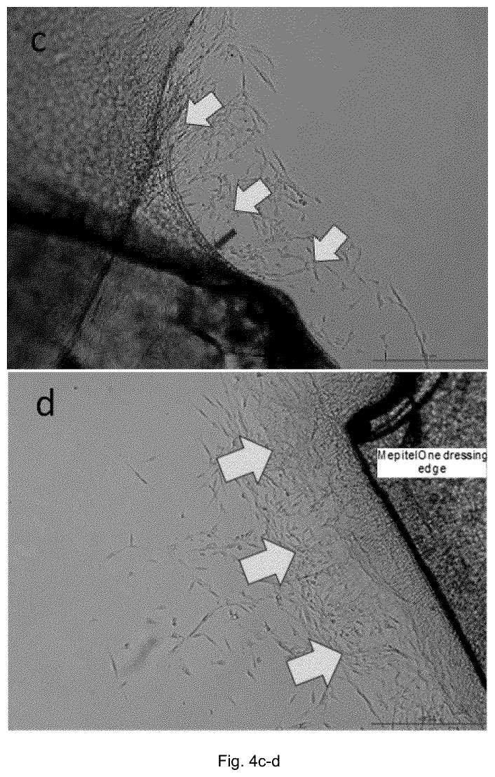

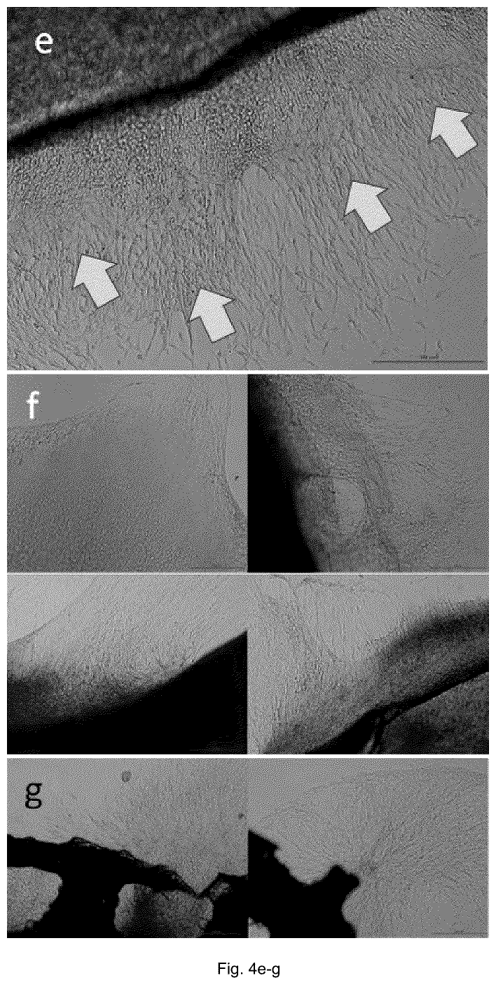

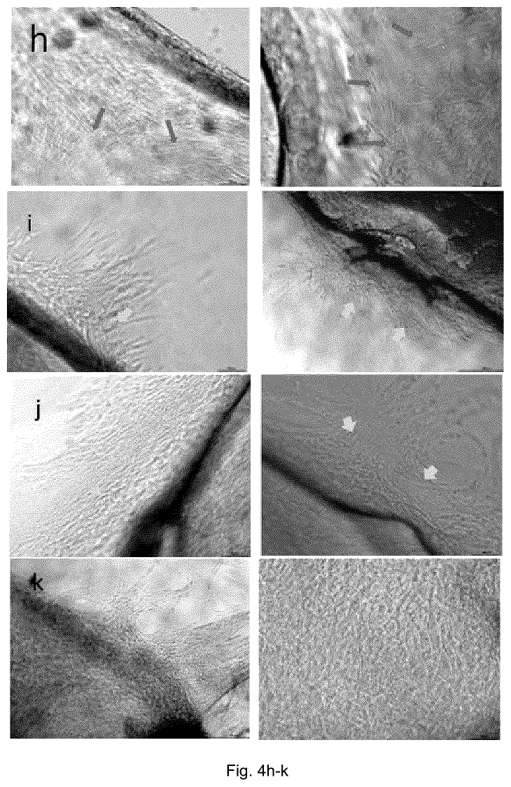

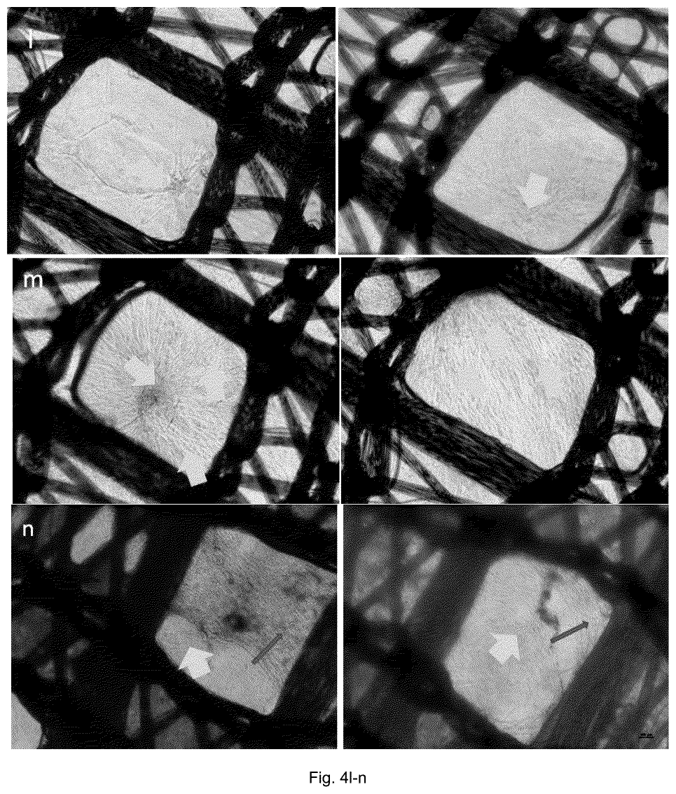

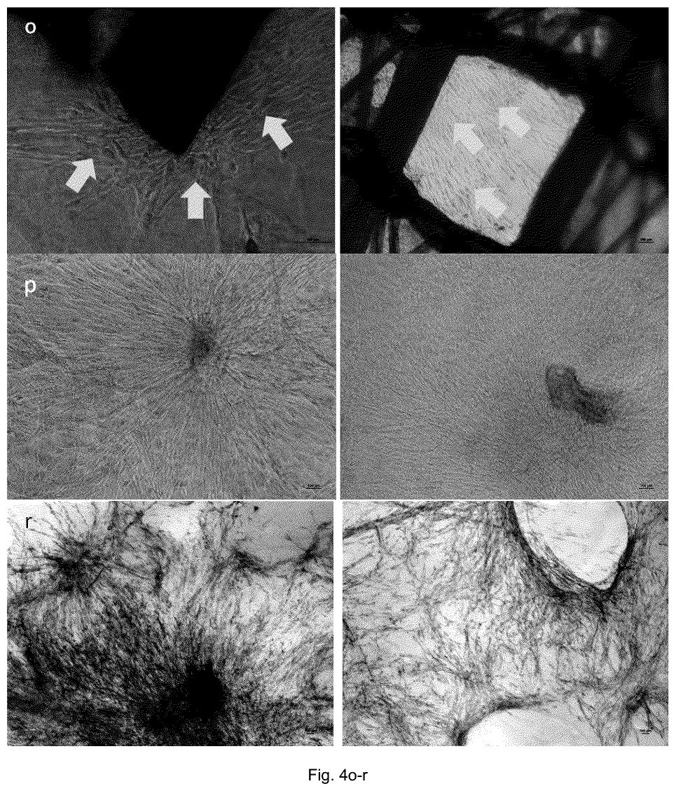

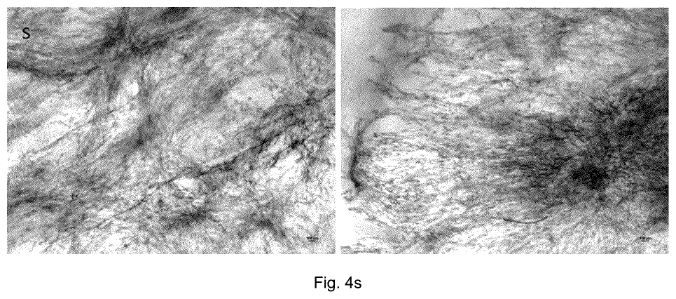

The results are shown in FIG. 4 which presents images based on microscope observations after the dressings were applied on the model wound environment. The narrow arrows indicate sites in which cells are still found on the dressing and the wide arrows indicate sites in which cells diffused from the dressing to the model wound environment which consists of fibrin glue mixed with a wound fluid. a-b—UrgoTul 3 days after the dressings were applied, c-d—MepitelOne 3 days after the dressings were applied, f—MepitelOne 7 days after the dressings were applied, g—UrgoTul 7 days after the dressings were applied, h—images of the MepitelOne dressing immediately after thawing and application on the model wound environment, day 1, i—images of the MepitelOne dressing 3 days after thawing and application on the model environment, j—images of the MepitelOne dressing 7 days after thawing and application on the model environment, k—images of the glue after removing the MepitelOne dressing from the model wound environment; position in which the edge of the dressing was located; sites of cell migration from the dressing can be seen, but the whole cell coat previously located on the dressing which remained on the glue after being removed from the model is also seen; and another image of the central location of the dressing on the glue (not the edge). l—Atrauman silicone 3 days after the dressing were applied, m—Atrauman silicone 7 days after the dressings were applied, n—images of the Atrauman silicone dressing 3 days after thawing and application on the model environment, o—images of the Atrauman silicone dressing 7 days after thawing and application on the model environment, p—images of the glue after removing the Atrauman silicone dressing from the model wound environment, sites of cell migration from the dressing can be seen, r—images of the glue after removing the Atrauman silicone dressing from the model wound environment, to visualize the site of cell migration from the dressing the cells were treated with MTT, formation of formazan crystals from MTT confirms cell viability (these procedures are based on the reduction of tetrazolium by mitochondrial dehydrogenase enzymes, which is carried inside living cells), s—images of the glue after removing the thawed Atrauman silicone dressing from the model wound environment, to visualize of site of cell migration from the dressing the cells were treated with MTT, formation of formazan crystals from MTT confirms cell viability (these procedures are based on the reduction of tetrazolium by mitochondrial dehydrogenase enzymes, which is carried inside living cells).

### EXAMPLE 5. WOUND HEALING ASSAY (“SCRATCH CLOSURE TEST”)

To confirm the therapeutic usefulness of the dressings selected in previous tests and obtained using the UrgoTul, Mepitel One and Atrauman silicone materials, they were subjected to an additional test in a wound healing assay using human fibroblast cells.

Preparation of Human Fibroblast Cells (nHF) for the Wound Healing Assay.

After storage in liquid nitrogen, nHF cells were thawed at 37° C. and transferred to Falcon tubes with an appropriate culture medium. The suspension was centrifuged at 5 min, 350×g, 22° C. After centrifugation, the supernatant was removed and a fresh volume of the culture medium previously heated to 37° C. was added to the remaining pellet; subsequently, their density was determined using an ADAM MC cell counter. The optimum density of nHF cells seeded in a T75 bottle is 0.3-0.8×10⁶ cells. The incubation conditions were maintained at 37° C. and 5% CO₂. The cells were cultured in a culture medium specific for the cells. Cultures were placed in an incubator and grown until the whole surface of the culture vessel was coated and then passaged or used for preparing an experiment. To this end, the medium should be removed, and cells washed with DPBS w/o Ca, Mg previously heated to 37° C. After removing DPBS, Accutase heated to room temperature is poured on the cells. The culture vessel with Accutase is placed at 37° C. for 5 minutes (incubation time with Accutase can be increased to 20 minutes, and the degree of cell detachment is tested every 3-5 minutes). To harvest the cells, an adequate volume of the growth medium is added to the culture vessel, pipetted several times and the whole culture suspension is collected into a sterile test tube. The cell suspension is centrifuged for 5 minutes at 350×g, 22° C. The supernatant is removed after centrifugation.

A fresh volume of the medium heated to 37° C. is added to the cell pellet and pipetted several times to obtain homogeneous cell suspension to be counted.

Dual chamber inserts were placed on a 24-well plate to provide even spaces for closure. Cells at a density of 1.5×10⁴ cells/insert well were seeded in the spaces between the inserts. The plate was placed in an incubator. The incubation conditions were maintained at 37° C. and 5% CO₂. The cells were cultured in a DMEM Low Glucose growth medium with 10% FBS (fetal bovine serum) and 1% of an antibiotic mix (penicillin and streptomycin). The cells used in the experiments were between passages 1 and 4.

### Wound Healing Test

Before and after the wound healing mimicking test, a Presto Blue assay was performed to evaluate/determine fibroblast cell metabolic activity. Subsequently, nHF cells and ADSCs on the dressings were stained with fluorescent dyes according to the manufacturer's protocol, and the dressings with the cells were immediately placed on the fibroblasts. The scratch assay is a laboratory technique used to analyze cell migration and cell-cell interactions. It is performed by creating a cell-free area e.g. by scratching a single cell layer or using inserts and recording images of the scratch space at regular time intervals. The scratch assay is dedicated for testing the migration potential of cells, such as e.g. fibroblasts that remodel and repair the connective tissue.

Through the analysis of images recorded during the experiment, the percentage closure level for the free space was determined. The free space is 100% at the initial stage. Any new cells that appear in the visual field confirm their migration and proliferation potential, which contributes to the percentage decrease of the space of the scratch being recorded. The microscope images of scratch closure were recorded using a Nikon Ti automated fluorescence microscope in the inverted configuration with a cell incubation chamber which maintained adequate environmental parameters (37° C., 5% CO₂).

The microscope operated in the PFS mode (Perfect Focus System). Fluorescent staining of the cells was performed with Vybrant Cell-Labeling Solutions dyes to differentiate the cells and to visualize them under the dressing, especially fibroblasts. For staining fibroblasts cells, a red dye was used and ADSCs were stained using a green dye. Images were recorded at 3 h intervals for 72 h.

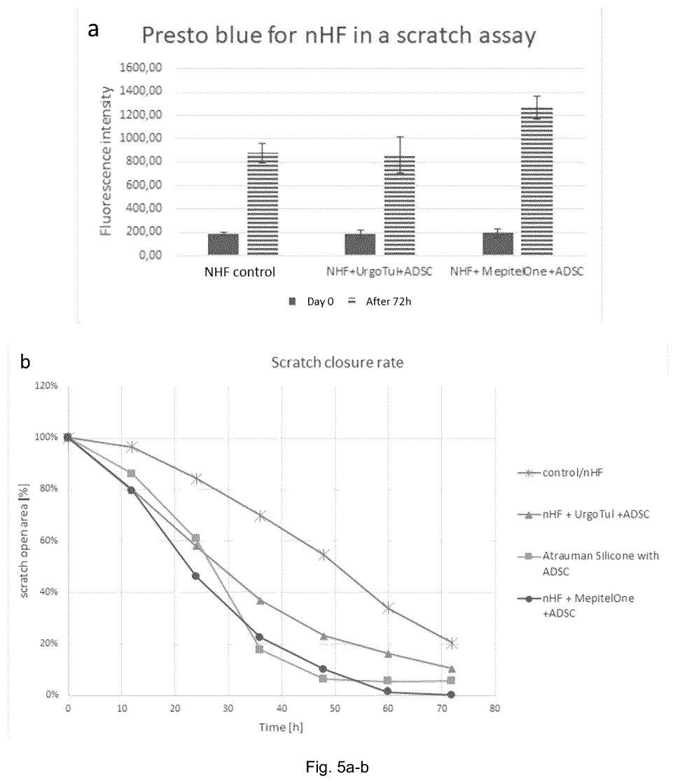

The experimental results for respective stages of the wound healing test are summarized in FIG. 5.

A positive effect on scratch closure was seen in the presence of the dressings obtained from ADSCs using the UrgoTul, MepitelOne and Atrauman silicone materials.

The scratch area of nHF cells cultured in the presence of ADSC seeded on UrgoTul material decreased to 10.3% after 72 h. While the scratch area was completely closed after 72 h in both variants where either dressing obtained from ADSC seeded on MepitelOne or on Atrauman silicone were used.

### EXAMPLE 6. LONG-TERM FREEZING IN LIQUID NITROGEN

To confirm the possibility of long-term storage of dressings prepared in the Example 1 ADSC were seeded on MepitelOne dressing material and stored frozen in liquid nitrogen accordingly to the protocol presented in Example 3. The dressings were stored in liquid nitrogen for 14 days and 54 days. Then the dressings were thawed and the ADSCs viability was tested with Presto Blue assay according to the protocol presented in Example 2. Unexpectedly the highest ADSC proliferation rate was observed on MepitelOne dressings stored for 54 days in liquid nitrogen. The results are presented in FIG. 6a. To confirm the presence of the viable cells at the first day of observation −24 h after thawing, the results were compared to the blank baseline. On the seventh day of the observation MepitelOne dressing stored frozen for 54 days showed very high fluorescence peak which might be the result of cells finally adapting to the culturing conditions after thawing. Even the dressings stored in liquid nitrogen for the period of 1 year showed visible live cells that populated the free space of the dressing in microscope observations. The results are shown in the FIG. 6b-c.

### EXAMPLE 7. COMPARATIVE EXAMPLE OF ADSC MIGRATION TEST FROM DRESSING WHICH WAS PREPARED IN ACCORDANCE WITH THE DESCRIPTION OF THE PATENT APPLICATION US 2018/0117217 IN A MODEL WOUND ENVIRONMENT

Model wound environment and experiment was performed according to Example 4.

Two dressings were prepared with Atrauman silicone dressing material: one of them according to the description of example 3 of the patent application US 2018/0117217, and another according to example 1.

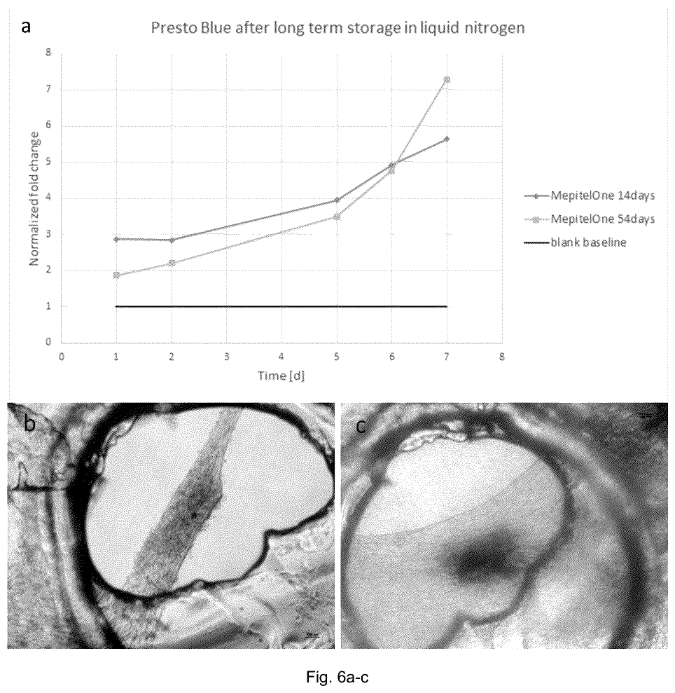

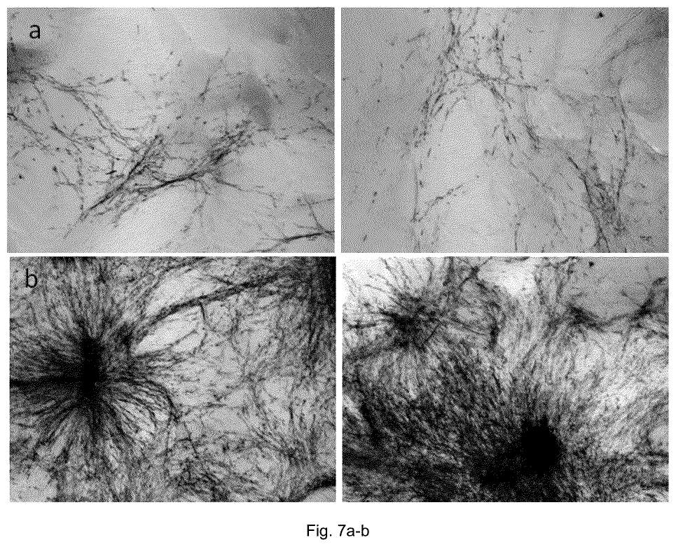

FIG. 7 presents images based on microscope observation after the dressings were applied on the model wound environment. The cells form the dressing from example 1 (our application) showed much higher migration than from the dressing prepared according to the patent application US 2018/0117217.

The cell migration to the wound environment is a vital trait of this invention and is a prerequisite to achieving the technical purpose thereof. The dressing prepared according to the patent application US 2018/0117217 is different and failed to fulfill requirements of the subject invention.

### REFERENCES

1. Bobis S, Jarocha D, Majka M. Mesenchymal stem cells: characteristics and clinical applications. Folia Histochem Cytobiol. 2006; 44:215-230.
2. Pittenger M F, Mackay A M, Beck S C et al. Multilineage potential of adult human mesenchymal stem cells. Science. 1999; 284:143-147.
3. Dominici M, Le Blanc K, Mueller I et al. Minimal criteria for defining multipotent mesenchymal stromal cells. The International Society for Cellular Therapy position statement. Cytotherapy. 2006; 8:315-317.
4. Witkowska-Zimny M, Walenko K. Stem cells from adipose tissue. Cell Mol Biol Lett. 2011; 16:236-257.
5. Chamberlain G, Fox J, Ashton B et al. Concise review: mesenchymal stem cells: their phenotype, differentiation capacity, immunological features, and potential for homing. Stem Cells. 2007; 25:2739-2749.
6. Wakitani S, Saito T, Caplan A I. Myogenic cells derived from rat bone marrow mesenchymal stem cells exposed to 5-azacytidine. Muscle Nerve. 1995; 18:1417-1426.
7. Woodbury D, Schwarz E J, Prockop D J et al. Adult rat and human bone marrow stromal cells differentiate into neurons. J Neurosci Res. 2000; 61:364-370.
8. Buzanska L, Jurga M, Stachowiak E K et al. Neural stem-like cell line derived from a nonhematopoietic population of human umbilical cord blood. Stem Cells and Development. 2006; 15:391-406.
9. Brzoska E, Grabowska I, Hoser G et al. Participation of stem cells from human cord blood in skeletal muscle regeneration of SCID mice. Exp Hematol. 2006; 34:1262-1270.
10. Ratajczak M Z, Kucia M, Reca R et al. Stem cell plasticity revisited: CXCR4-positive cells expressing mRNA for early muscle, liver and neural cells ‘hide out’ in the bone marrow. Leukemia. 2004; 18:29-40.

1. A dressing for use in treating ulceration, in particular the diabetic foot, wherein said dressing consists of:
a dressing material and
ADSCs embedded therein,
wherein the dressing material consists of a flat substrate having holes and an adhesive layer that coats its surface,
wherein the flat substrate is made of a polymer containing polyester or polyurethane,
while the adhesive layer contains a substance selected among of: silicone gel and a hydrocolloid containing carboxymethylcellulose or its salts with alkaline metals dispersed in a matrix containing petrolatum and paraffin oil.

2. A dressing for use of claim 1, characterized in that it contains at least 1.73×10⁵ ADSCs per 1 cm² substrate area.

3. A dressing for use of claim 1, characterized in that the mean hole size in the substrate up to 1300 μm.

4. A dressing for use of claim 1, characterized in that the substrate is in the form of a mesh or membrane.

5. A dressing for use of claim 1, characterized in that the adhesive layer is coated with extracellular matrix proteins selected among of: fibronectin, collagen, laminin, elastin and vitronectin, possibly prior to cell seeding to enhance cell adhesion.

6. A dressing for use of claim 1, characterized in that ADSCs are unstimulated ADSCs.

7. A dressing for use of claim 1, characterized in that ADSCs are seeded on the dressing material.

8. A method for the manufacture of a dressing for use in treating ulceration, in particular the diabetic foot, characterized in that it includes stages in which:
a) the dressing material is coated with fibronectin,
b) ADSC suspension in a growth medium is applied on the surface of the fibronectin-coated dressing material,
c) the cells are grown on the dressing material,
d) the dressing material with the embedded ADSCs is separated and optionally stored frozen,
wherein a dressing material is used that
consists of a flat substrate having holes and an adhesive layer that coats its surface, wherein the flat substrate is made of an organic polymer containing polyester or polyurethane,
while the adhesive layer contains a substance selected among of: silicone gel and a hydrocolloid containing carboxymethylcellulose or its salts with alkaline metals dispersed in a matrix containing petrolatum and paraffin oil.

9. A method of claim 8, characterized in that in stage a) the dressing material is immersed for 30 to 60 minutes in fibronectin solution being a mixture of fibronectin in DPBS w/o Ca, Mg, preferably at a 1:100 ratio.

10. A method of claim 8, characterized in that in stage b) the ADSC suspension in the XenoFree medium is applied with cell density between 1.25×10⁶ and 4.0×10⁶ cells/mL.

11. A method of claim 8, characterized in that in stage b) 200 μL of the ADSC suspension in the XenoFree medium with cell density of 1.25× 10⁶ cells/mL is applied on a dressing with a size of 1.2 cm×1.2 cm.

12. A method of claim 8, characterized in that in stage c) the cells are cultured in the XenoFree medium intended for culturing human mesenchymal stem cells for at least 3 hours at 37° C. and 5% CO₂.

13. A method of claim 8, characterized in that in stage d) the dressing material with the ADSCs embedded therein is frozen at −80° C. for at least 24 h, and subsequently stored in liquid nitrogen.

14. A method of claim 10, characterized in that in stage b) 200 μL of the ADSC suspension in the XenoFree medium with cell density of 1.25×10⁶ cells/mL is applied on a dressing with a size of 1.2 cm×1.2 cm.
